(19) United States
(12) Patent Application Publication
(10) Pub. No.: US 2020/0253772 A1
(43) Pub. Date: Aug. 13, 2020

(54) Waterproof Ankle-Foot Orthosis

(71) Applicants: Mary-Kate Reid, Bedford, MA (US); Michael Gallagher Reid, Bedford, MA (US)

(72) Inventors: Mary-Kate Reid, Bedford, MA (US); Michael Gallagher Reid, Bedford, MA (US)

(21) Appl. No.: 16/784,014

(22) Filed: Feb. 6, 2020

Related U.S. Application Data
(60) Provisional application No. 62/802,485, filed on Feb. 7, 2019.

(51) Int. Cl. A61F 5/01; A61F 2/50; A61F 2/78

(52) U.S. Cl. CPC A61F 5/0127; A61F 2/5046; A61F 2002/5089; A61F 2002/7806; A61F 2002/785; A61F 2005/0132

(57) ABSTRACT

An ankle-foot orthosis which is capable of being worn by a user in an acquatic environment which is lightweight, water-resistant, and reduces the amount of hydrodynamic drag during motion of a leg of a user when in water. The ankle-foot orthosis can be fully customizable to the needs of the user. The ankle-foot orthosis including three protective components; a shank shell, a shin plate, and a foot shell. The ankle-foot orthosis optionally including an additional protective component; a dorsal foot plate. At least one flow passage within each of the shank shell and the foot shell. The at least one flow passage providing water drainage and air ventilation, and reducing the overall weight of the shank shell and foot shell.

### CROSS REFERENCE TO RELATED APPLICATIONS

This application claims the benefit of and priority under 35 U.S.C. § 119(e) to U.S. Provisional Patent Application No. 62/802,485, filed Feb. 7, 2019, which is incorporated by reference herein in its entirety.

### TECHNICAL FIELD

This application relates generally to medical devices. In particular, this application relates to ankle-foot orthoses which can be used in an aquatic environment.

### BACKGROUND

An ankle foot orthosis (“AFO”) can be used to provide support and assistance to a user throughout the gait cycle. AFOs are also used for controlling the position and motion of an ankle of the user. There are various types of AFOs; some which comprise a solid ankle with no ankle joint and others with ankle joints which provide the ability to adjust the ankle position. AFOs are utilized based on the needs of the user. Some users require the assistance of an AFO only at night and others require the assistance throughout the day and night. AFOs are frequently worn by users who require support and protection of the lower legs and ankles due to various disorders.

Current designs of AFOs are not recommended to be used in an aquatic environment. They are made of materials which either degrade from exposure to water, or if they are made from water-resistant materials they are not designed in a manner to permit adequate mobility of the user in the water. Due to the current designs, users may opt not to wear an AFO in the water. However, users are at as much risk of injury in the water as they are on land.

### SUMMARY

Accordingly, a need currently exists for an AFO which is capable of being worn not only on land, but also in water. An AFO compatible for use in water needs to be lightweight, water-resistant, and reduce the amount of hydrodynamic drag during motion of the leg of the user when in the water.

In one aspect, an ankle-foot orthosis is featured including a shank shell, a shin plate, and a foot shell. The shank shell, shin plate, and foot shell each comprising an outer rigid shell and an inner padding. The shank shell and the foot shell each containing at least one flow passage. The shank shell and the foot shell are permanently connected by a flexure joint. The shank shell is secured to the leg of the user through a releasable shank ratchet system. The shin plate is attachable to the shank shell through the releasable shank ratchet system. The foot shell is secured to the foot of the user through an adjustable dorsal foot buckle which is affixed to the foot shell.

In some embodiments, the shin plate includes at least one flow passage. The at least one flow passage on each of the shank shell, shin plate, and foot shell can include a flow-through mesh lining.

In some embodiments, the ankle-foot orthosis includes a dorsal foot plate. It can have an adjustable dorsal foot buckle configured to secure the dorsal foot plate to the foot shell. In some embodiments, the foot shell further comprises a tread affixed to a bottom of the foot shell for enhanced traction. An adhesive can secure the tread to the foot shell. In some embodiments, the outer rigid shell is formed of a semi-rigid water-resistant material. The inner padding can be formed of a pliable, lightweight, and water-resistant material. An adhesive joins the semi-rigid water-resistant material of the outer rigid shell to the pliable, lightweight, and water resistant material of the inner padding.

The invention, in another aspect, includes a method of manufacturing an ankle-foot orthosis. The method includes creating a mold of a lower leg of a user and creating a mold of a foot of the user. The mold is used to create a hardened outer rigid shell. An inner pad can be secured to an inner surface of the outer rigid shell using an adhesive. Flow passages can be created by removing material from the shank shell, and/or by removing material from a foot shell. A shank ratchet system can be mounted to the shank shell, and a shin plate can be attached to the shank shell. A dorsal foot buckle and a dorsal foot buckle sheath can be mounted to the foot shell.

In some embodiments, the ankle-foot orthosis can be manufactured to meet the individual needs of a particular user. The mold can be further tailored by the inclusion of a dorsal foot plate, and the dorsal foot plate can be attached to the foot shell using the dorsal foot buckle and the dorsal foot buckle sheath.

In some embodiments, the mold can be tailored by the addition of a plurality of flow passages to create a specific flow through area specific to the needs of the user. At least one flow passage should be included in the shin plate. In a further aspect, the flow passages can comprise a flow-through mesh material.

### BRIEF DESCRIPTION OF THE DRAWINGS

The advantages of the systems and methods described above, together with further advantages, may be better understood by referring to the following detailed description taken in conjunction with the accompanying drawings. The drawings are not necessarily to scale, emphasis instead generally being placed upon illustrating the principles of the described embodiments by way of example only.

FIG. 1 illustrates an example of a known dynamic ankle foot orthosis.

FIG. 2 is an illustrative example of a side view of a shank shell, foot shell, flexure joint, and dorsal foot buckle according to some embodiments disclosed herein.

FIG. 3 is an illustrative example of a side view of an ankle foot orthosis according to some embodiments disclosed herein.

FIG. 4 is an illustrative example of a side view of a shank shell according to some embodiments disclosed herein.

FIG. 5 is an illustrative example of a side view of a shin plate according to some embodiments disclosed herein.

FIG. 6 is an illustrative example of a side view of a dorsal foot plate according to some embodiments disclosed herein.

FIG. 7 is an illustrative example of a side view of a foot shell according to some embodiments disclosed herein.

FIG. 8 is a flow diagram of a method for manufacturing an ankle foot orthosis according to some embodiments disclosed herein.

### DETAILED DESCRIPTION

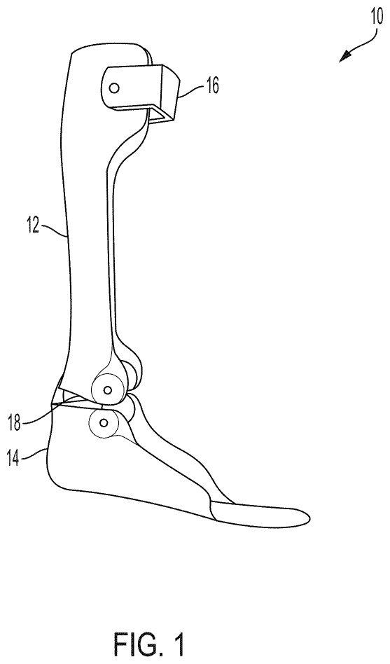

FIG. 1 illustrates an example of a known dynamic ankle-foot orthosis (“AFO”) 10. The dynamic AFO 10 includes a leg brace 12 and a foot brace 14. The leg brace 12 is secured to the leg of the user through a hook and loop strap or adhesive strip 16 around the shin of the user. The leg brace 12 encloses the calf of the user but leaves the shin of the user exposed. The foot brace 14 encloses the heel, ankle, sole, and sides of the foot of the user but leaves the bridge of the foot of the user exposed. The leg brace 12 and the foot brace 14 are typically made of materials that are not water compatible. The leg brace 12 and the foot brace 14 are connected by flexible hinge 18 and another flexible hinge (not shown) on the opposite side of flexible hinge 18. Flexible hinge 18 provides support for instability and control of the ankle and foot of the user.

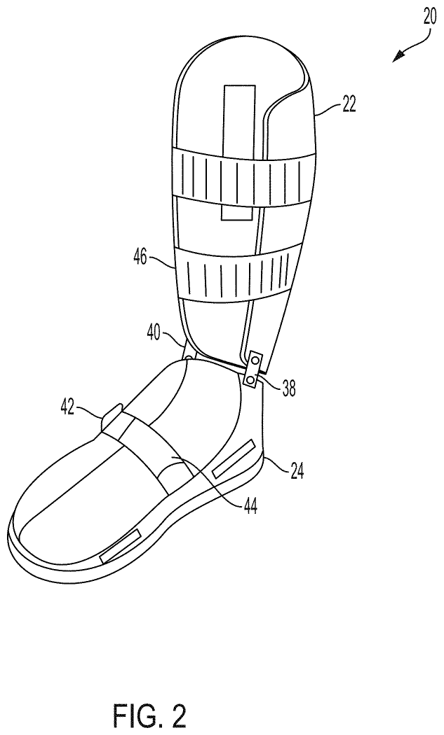

FIG. 2 is an illustrative example of a side view of an ankle-foot orthosis 20 according an embodiment of the invention. The AFO 20 includes two protective components; a shank shell 22 and a foot shell 24.

The shank shell 22 encloses the calf of the user to provide support and protection. The foot shell 24 encloses the heel, ankle, sole, and sides of the foot of the user to provide support and protection. The shank shell 22 and the foot shell 24 are connected by flexure joints 38 and 40. The flexure joints 38 and 40 serve to support the ankle while providing controlled dorsiflexion and plantarflexion mobility and prevent the ankle from over-extending into dangerous positions. In preferred embodiments the flexure joints 38, 40 are Tamarack Free Motion Ankle Joints.

At least one adjustable dorsal foot buckle 42 is connected to the foot shell 24 and secures the foot of the user to the foot shell 24 and the AFO 20. The dorsal foot buckle 42 also includes a dorsal foot buckle sheath 44. The adjustable dorsal foot buckle 42 can be a load-rated slip-on-buckle which is sufficiently strong to provide the security the user needs while also being inexpensive. The dorsal foot buckle sheath 44 can be made of a pliable polyester mesh or a similar material which is lightweight, breathable, flexible, and water resistant. The adjustable dorsal foot buckle 42 can provide the AFO 20 with the look that the user is wearing a traditional shoe along with the AFO 20 when in fact the user is not wearing a traditional shoe.

At least one shank ratchet system 46 is connected to the shank shell 22 and secures the leg of the user to the shank shell 22 and the AFO 20. The shank ratchet system 46 is sufficiently strong to secure the AFO 20 to the leg of the user without risk of failure. Additionally, the shank ratchet system 46 reduces the amount of time it takes to remove and attach the AFO 20 due to the shank ratchet system's 46 ease of use. The surface of the shank ratchet system (not shown) which touches the skin of the leg of the user can be lined with a pliable polyester mesh or a similar material which is lightweight, breathable, flexible, and water resistant. The polyester mesh attached to the surface of the shank ratchet system (not shown) is meant to provide comfort to the user.

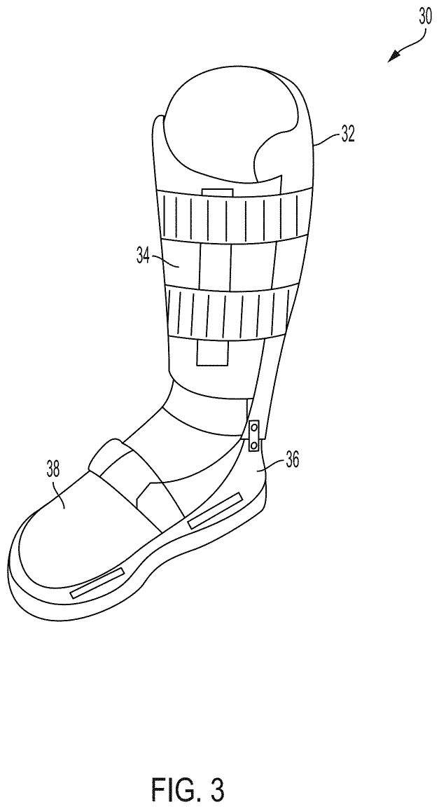

FIG. 3 is an illustrative example of a side view of an ankle-foot orthosis 30 according to some embodiments disclosed herein. The AFO 30 can include all the components as described in FIG. 2. The AFO 30 includes four protective components; a shank shell 32, a shin plate 34, a foot shell 36, and a dorsal foot plate 38. This is an illustrative example of an assembled AFO 30 which includes components that are described herein.

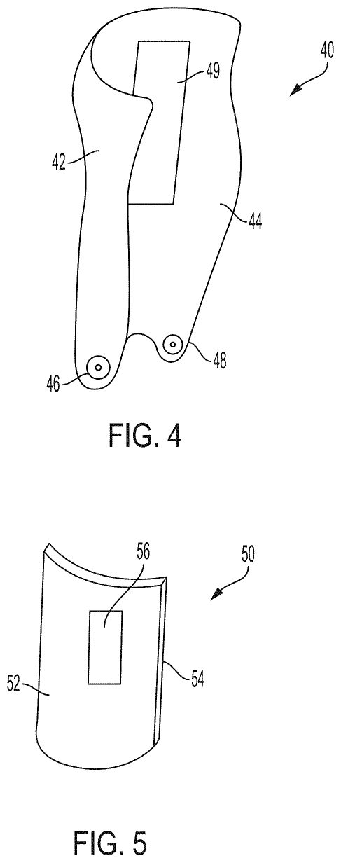

FIG. 4 is an illustrative example of a side view of a shank shell 40 according to some embodiments disclosed herein. The shank shell includes an outer rigid surface 42 and an inner padding 44. The outer rigid surface 42 can be made of a water-resistant thermoplastic polymer, for example, polypropylene. The inner padding 44 can be made of a pliable polyester mesh or a similar material which is lightweight, breathable, flexible, and water-resistant. The inner padding 44 can be 15 mm in thickness and 650 g in weight. One function of the inner padding 44 is to provide comfort to the user. Many users experience discomfort from their skin rubbing against certain materials, and the inner padding 44 reduces this discomfort.

The outer rigid surface 42 and the inner padding 44 can be joined by an adhesive and sealant (not shown). The adhesive and sealant have a temperature rating of between −65 degrees Celsius to 150 degrees Celsius. This encompasses the temperature range in which the user would use the AFO, e.g., as shown in FIG. 2 and FIG. 3. The adhesive and sealant is effective against salt water and highly flexible to bond materials with different thermal expansions.

The shank shell 40 can have impressions 46 and 48 where the flexure joints (not shown) attach to in order to connect the shank shell 40 and the foot shell (not shown).

There is at least one flow passage 49 formed in the shank shell 40. The flow passage 49 can have different sizes and shapes. For example, the flow passage 49 can be a rectangular slit as shown. Alternatively, the shape of the flow passage 49 can be e.g., a circle or star shaped. A circular shape can be easily manufactured. A child user may want to personalize their AFO by having star shaped flow passages. That is, the functional opening can include ornamental shapes to appeal to a younger user. The flow passage 49 has a minimum flow through area of 15% and a maximum flow through area of up to 40% based on a cross-sectional frontal area of the shank shell 40. A purpose of the flow passage 49 is to provide water drainage when the AFO, as shown in FIG. 2 and FIG. 3, facilitating use and mobility in an aquatic environment. Water drainage allows safer usage of the AFO, as shown in FIG. 2 and FIG. 3, in an aquatic environment since the overall weight of the AFO is reduced. Another purpose of flow passage 49 is to provide air ventilation and additional comfort when the AFO, as shown in FIG. 2 and FIG. 3, is used in a warm environment. Yet another purpose of flow passage 49 is to reduce the overall weight of the AFO, and to reduce hydraulic drag as the user navigates an aquatic environment.

FIG. 5 is an illustrative example of an optional side view of a shin plate 50 according to some embodiments disclosed herein. Not all users will require the shin plate. As shown, the shin plate 50 includes an outer rigid surface 52 and an inner padding 54. The outer rigid surface 52 can be made of a water-resistant thermoplastic polymer, for example, polypropylene. The inner padding 54 can be made of a pliable polyester mesh or a similar material which is lightweight, breathable, flexible, and water resistant. The inner padding 54 can be 15 mm in thickness and 650 g in weight. One function of the inner padding 54 is to provide comfort to the user. Many users experience discomfort from their skin rubbing against certain materials, and the inner padding 54 reduces this discomfort.

The outer rigid surface 52 and the inner padding 54 can be joined by an adhesive and sealant (not shown). The adhesive and sealant should have a temperature rating of between about −65 degrees Celsius and 150 degrees Celsius. This encompasses the temperature range in which the user would use the device. The adhesive and sealant is effective against salt water and highly flexible to bond materials with different thermal expansions.

Optionally, there can be at least one flow passage 56 formed in the shin plate 50. The flow passage 56 can have a variety of different sizes and shapes. For example, the flow passage 56 can be a rectangular slit as shown. The user can customize the shape of the flow passage 49 and make it for example, a circle or star shaped. A circular shape can be easily manufactured. A child user may want to personalize their AFO by having star shaped flow passages. That is, the functional opening can include ornamental shapes to appeal to a younger user. A purpose of the flow passage 56 is to provide water drainage when the AFO, as shown in FIG. 2 and FIG. 3, is used in an aquatic environment. Water drainage allows safer usage and better mobility of the AFO, as shown in FIG. 2 and FIG. 3, in an aquatic environment since the overall weight of the AFO is reduced. Another purpose of flow passage 56 is to provide air ventilation and additional comfort when the AFO, as shown in FIG. 2 and FIG. 3, is used in a warm environment. Yet another purpose of flow passage 56 is to reduce the overall weight of the AFO, and to reduce hydraulic drag as the user navigates an aquatic environment.

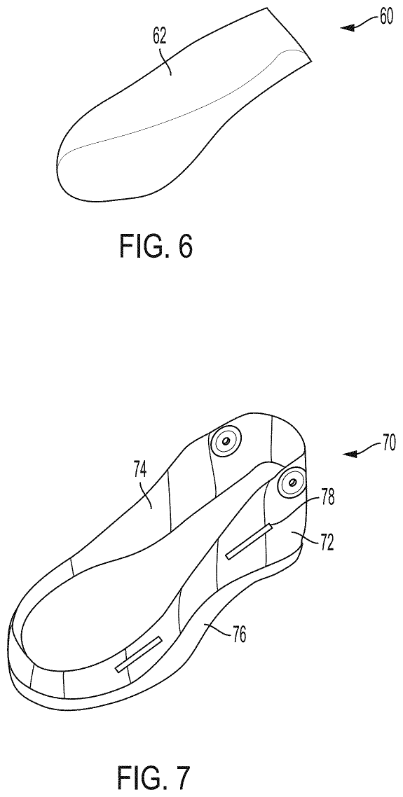

FIG. 6 is an illustrative example of a side view of a dorsal foot plate 60 according to another embodiment of the invention. The dorsal foot plate 60 can include an outer rigid surface 62 and an inner padding (not shown). The outer rigid surface 62 can be made of a water-resistant thermoplastic polymer, for example, polypropylene. The inner padding (now shown) can be made of a pliable polyester mesh or similar material which is lightweight, breathable, flexible, and water resistant. The inner padding (not shown) can be 15 mm in thickness and 650 g in weight. One function of the inner padding (not shown) is to provide comfort to the user while not being forced to wear a traditional shoe along with the AFO, as shown in FIG. 2 and FIG. 3. The outer rigid surface 62 and the inner padding (not shown) are joined by an adhesive and sealant (not shown). The adhesive and sealant has a temperature rating of between −65 degrees Celsius to 150 degrees Celsius. This encompasses the temperature range in which the user would use the device. The adhesive and sealant is effective against salt water and highly flexible to bond materials with different thermal expansions.

Optionally, there can be at least one flow passage (not shown) formed in the dorsal foot plate 60. The flow passage (not shown) can have a variety of different sizes and shapes. The user can customize the shape of the flow passage (not shown) and make it for example, a rectangular slit, circle or star shaped. A purpose of the flow passage (not shown) is to provide water drainage when the AFO, as shown in FIG. 2 and FIG. 3, is used in an aquatic environment. Water drainage allows safer usage and better mobility of the AFO, as shown in FIG. 2 and FIG. 3, in an aquatic environment since the overall weight of the AFO is reduced. Another purpose of flow passage (not shown) is to provide air ventilation and additional comfort when the AFO, as shown in FIG. 2 and FIG. 3, is used in a warm environment. Yet another purpose of flow passage (not shown) is to reduce the overall weight of the AFO, and to reduce hydraulic drag as the user navigates an aquatic environment.

FIG. 7 is an illustrative example of a side view of a foot shell 70 according to some embodiments disclosed herein. The foot shell 70 includes an outer rigid surface 72 and an inner padding 74. The outer rigid surface 72 can be made of a water-resistant thermoplastic polymer, for example, polypropylene. The inner padding 74 can be made of a pliable polyester mesh or similar material which is lightweight, breathable, flexible, and water-resistant. The inner padding 74 can be 15 mm in thickness and 650 g in weight. One function of the inner padding 74 is to provide comfort to the user while not being forced to wear a traditional shoe along with the AFO, as shown in FIG. 2 and FIG. 3. On the bottom of the foot shell 70, tread 76 can be affixed for enhanced traction. This enhanced traction can be specifically useful when the user is wearing the AFO, as shown in FIG. 2 and FIG. 3, in an aquatic environment.

There is at least one flow passage 78 within the foot shell 70. The flow passage 78 can have a variety of different sizes and shapes. For example, the flow passage 78 can be a rectangular slit. The user can customize the shape of the flow passage 78 and make it for example, a circle or star shaped. The flow passage 78 has a minimum flow through area of 5% and a maximum flow through area of 20% of a cross-section of the foot shell 70. A purpose of the flow passage 78 is to provide water drainage when the AFO, as shown in FIG. 2 and FIG. 3, is used in an aquatic environment. Water drainage allows safer usage of the AFO, as shown in FIG. 2 and FIG. 3, in an aquatic environment since the overall weight of the AFO is reduced. Another purpose of flow passage 78 is to provide air ventilation and additional comfort when the AFO, as shown in FIG. 2 and FIG. 3, is used in a warm environment.

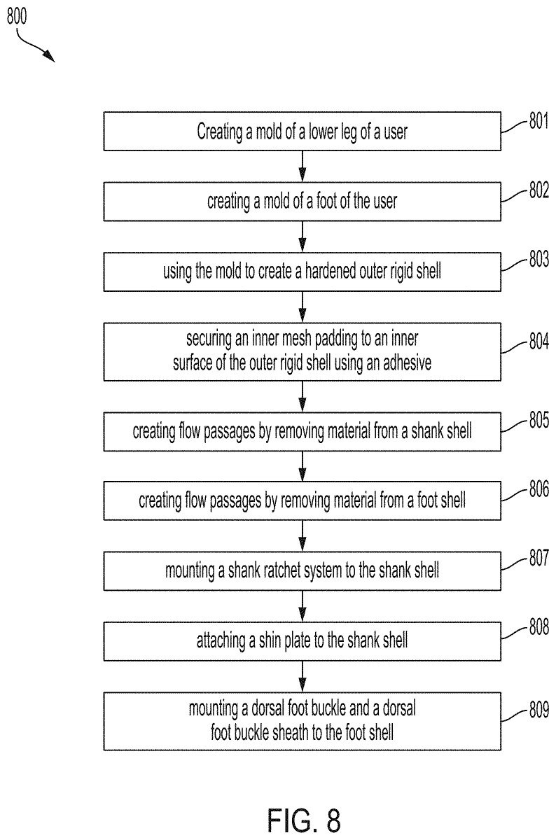

FIG. 8 is a flow diagram 800 of a method for manufacturing an ankle foot orthosis according to some embodiments disclosed herein. As shown in FIG. 8, manufacturing an ankle foot orthosis can include creating a mold of a lower leg of a user (step 801), creating a mold of a foot of the user (step 802), and using the mold to create a hardened outer rigid shell (step 803). Once the hardened outer rigid shell is created, securing an inner padding to an inner surface of the outer rigid shell using an adhesive (step 804), creating flow passages by removing material from a shank shell (step 805), and creating flow passages by removing material from a foot shell (step 806). Mounting a shank ratchet system to the shank shell (step 807), attaching a shin plate to the shank shell (step 808), and mounting a dorsal foot buckle and a dorsal foot buckle sheath to the foot shell (step 809).

At step 801 and 802, creating a mold of both the lower leg and the foot of the user allows for the AFO, as shown in FIG. 1 and FIG. 2, to be customized to the physical dimensions of the user. Alternatively, one mold can be made of the lower leg and foot which is subsequently cut into around the ankle to create two pieces. At step 801, a mold of a shin plate is also created. At step 803, flexure joint replicas are fastened onto the mold, one replica on each ankle, so that the midpoint of the flexure joint replicas are located on the ankle axis. These flexure joint replicas create a cavity in the outer rigid shell where the flexure joints will eventually be attached later in the manufacturing process. The mold is wrapped with a thermoplastic which has been heated to a pliable forming temperature. The mold is wrapped at last once but can be wrapped more than once in order to reinforce certain areas of the mold. After the mold is wrapped, the mold and heated thermoplastic are cooled to solidify. This creates the hardened outer rigid shell. A standard trim line is created and the outer rigid shell is trimmed based on the trim line. The standard trim line extends from the fibular head to the bottom of the foot, leaving the shin and bridge of foot area exposed. This trimming can be done by cutting along the trim line with an oscillating saw or any other similar cutting method. Once the trimming is complete, the hardened outer rigid shell provides no protection around the shin and bridge of the foot. The width of the material that was removed from the outer rigid shell should be less than the width of the shin plate. Alternatively, the trim line can remove only a slit from the shin area, so that the outer rigid shell encloses the shin area as well. This would eliminate a shin plate because the shin area would already be protected by the shank shell. The mold is removed from the hardened outer rigid shell, which creates the hollow ankle-foot orthosis structure.

At step 804, an inner padding is secured to an inner surface of the outer rigid shell using an adhesive and sealant. The adhesive and sealant has a temperature rating of between −65 degrees Celsius to 150 degrees Celsius. This encompasses the temperature range in which the user would use the device. The adhesive and sealant is effective against salt water and highly flexible to bond materials with different thermal expansions.

At steps 805 and 806, flow passages are created in both the shank shell and foot shell by removing material from the shank shell and foot shell respectively. This removing of material can be done by cutting the outline of the designed flow passages in the shank shell and foot shell with an oscillating saw or any other similar cutting method. After the flow passages have been created the edges of the trim line and flow passages can be smoothed by a finishing process.

At step 807, at least one shank ratchet system is mounted to the shank shell by riveting the components onto the shank shell. Instead of riveting the components, similar securing methods may be used. The flexure joints are inserted into the cavities created in step 803 and are secured with metal fasteners and anchoring screws. Instead of metal fasteners and anchoring screws, similar securing methods may be used. At step 808, a shin plate is attached to the shank shell by securing the shin plate between the shank ratchet system and shank shell. At step 809, at least one dorsal foot buckle and dorsal foot buckle sheath are mounted to the foot shell. The dorsal foot buckle and dorsal foot buckle sheath are mounted by cutting a slot into the foot shell on either sides of the foot. The dorsal foot buckle sheath is inserted through the slots and fixed to the foot shell with a rivet on either sides.

Instead of riveting the components, similar securing methods may be used. The dorsal foot buckle is then inserted through the dorsal foot buckle sheath.

To provide further protection to the foot of the user, a dorsal foot plate can be created by the manufacturing process as described in steps 801-804. The dorsal foot plate can be attached to the foot shell using the dorsal foot buckle and dorsal foot buckle sheath to secure it.

The user can customize the mold at steps 805 and 806 by the addition of a plurality of flow passages to create a specific flow through area to meet their particular needs. At least one flow passage can be created in the dorsal foot plate. This at least one flow area can be used for water drainage and air ventilation. At least one flow passage can be created in the shin plate. This at least one flow area can be used for water drainage and air ventilation. The flow through passages can also comprise a flow-through mesh material.

While the invention has been particularly shown and described with reference to specific illustrative embodiments, it should be understood that various changes in form and detail may be made without departing from the spirit and scope of the invention.

What is claimed is:

1. An ankle-foot orthosis for protecting and supporting a leg of a user, the ankle-foot orthosis comprising:
a shank shell configured to provide support and protection to the leg of the user, the shank shell comprising an outer rigid shell, an inner padding, and at least one flow passage;
a shin plate configured to provide support and protection to the leg of the user, the shin plate attachable to the shank shell, the shin plate comprising an outer rigid shell and an inner padding;
a foot shell configured to provide support and protection to a foot of the user, the foot shell comprising an outer rigid surface, an inner padding, and at least one flow passage;
a flexure joint permanently connecting the shank shell and the foot shell, the flexure joint configured to provide controlled dorsiflexion and plantarflexion mobility and protection from over extension;
a releasable shank ratchet system configured to progressively secure the shank shell and the shin plate relative to the leg of the user during fitting of the ankle-foot orthosis to the leg of the user; and
an adjustable dorsal foot buckle affixed to the foot shell, the adjustable dorsal foot buckle configured to secure the foot shell while comfortably providing support to the foot of the user.

2. The ankle foot orthosis of claim 1, wherein the shin plate has at least one flow passage.

3. The ankle foot orthosis of claim 1, wherein the at least one flow passage through the shank shell has a minimum flow through area of 15% and a maximum flow through area of 40% of a cross-section of the shank shell.

4. The ankle foot orthosis of claim 1, wherein the at least one flow passage through the foot shell creates a minimum flow through area of 5% and a maximum flow through area of 20% of a cross-section of the shank shell.

5. The ankle foot orthosis of claim 1, further comprising a dorsal foot plate configured to provide support and protection to the foot of the user, the dorsal foot plate comprising an outer rigid surface and an inner padding.

6. The ankle foot orthosis of claim 5, wherein the outer rigid shell of the dorsal foot plate is formed of a semi-rigid water-resistant material.

7. The ankle foot orthosis of claim 5, wherein the inner padding of the dorsal foot plate is formed of a pliable water-resistant material.

8. The ankle foot orthosis of claim 5, wherein the adjustable dorsal foot buckle is configured to secure the dorsal foot plate to the foot shell.

9. The ankle foot orthosis of claim 1, wherein the foot shell further comprises a tread affixed to a bottom of the foot shell for enhanced traction.

10. The ankle foot orthosis of claim 1, wherein an adhesive joins the outer rigid surfaces and the inner paddings.

11. The ankle foot orthosis of claim 1, wherein the at least one flow passage comprises a flow-through mesh lining.

12. The ankle foot orthosis of claim 1, wherein the outer rigid shell of the shank shell, the shin plate, and the foot shell are formed of a semi-rigid water-resistant material.

13. The ankle foot orthosis of claim 1, wherein the inner padding of the shank shell, the shin plate, and the foot shell are formed from a pliable water-resistant material.

14. A method of manufacturing an ankle-foot orthosis, the method comprising:
creating a mold of a lower leg of a user;
creating a mold of a foot of the user;
using the mold to create a hardened outer rigid shell;
securing an inner padding to an inner surface of the outer rigid shell using an adhesive;
creating flow passages by removing material from the shank shell;
creating flow passages by removing material from the foot shell;
mounting a shank ratchet system to the shank shell;
attaching the shin plate to the shank shell and
mounting a dorsal foot buckle and a dorsal foot buckle sheath to the foot shell.

15. The method claim of 14, wherein the mold of the lower leg and foot of the user is created by one mold which is further cut into two pieces.

16. The method claim of claim 14, wherein the mold is further tailored by the inclusion of a dorsal foot plate.

17. The method claim of claim 14, wherein the mold is further tailored by the addition of a plurality of flow passages to create a specific flow through area.

18. The method claim of claim 14, wherein a dorsal foot plate is attached to the foot shell using the dorsal foot buckle and the dorsal foot buckle sheath.

19. The method claim of claim 14, wherein material is removed from the shin plate to create at least one flow passage.

20. The method claim of claim 14, wherein the flow passages comprise a flow-through mesh material.
